(19) United States
(12) Patent Application Publication
(10) Pub. No.: US 2010/0285128 A1
(43) Pub. Date: Nov. 11, 2010

(54) SPACE FILLED DRUG RELEASE STRUCTURE AND METHOD OF MANUFACTURING THE SAME

(76) Inventors: Wei-Hsing TUAN, Taipei City (TW); Shu-Ting Kuo, Taipei City (TW); Ting-Yu Tuan, Taipei City (TW); Tun-Fu Shih, Taipei City (TW); Yi-Ting Hsieh, Taipei County (TW)

Correspondence Address: Muncy, Geissler, Olds & Lowe, PLLC, 4000 Legato Road, Suite 310, FAIRFAX, VA, 22033, US

(21) Appl. No.: 12/539,271

(22) Filed: Aug. 11, 2009

(30) Foreign Application Priority Data
May 6, 2009 (TW) 098114937

(51) Int. Cl. A61K 9/00; A61K 33/42; A61P 43/00

(52) U.S. Cl. 424/484; 424/602

(57) ABSTRACT

A space filled drug release structure and a method of manufacturing the same are disclosed. The structure is composed of a highly water-absorbing polymeric matrix and drug particles dispersed in the matrix. The matrix has pores and passages interconnecting the pores together. Because some water is present in the matrix, gastric acid can easily penetrate into the matrix to reach each drug particle. The surface of each drug particle is therefore possible to contact with the gastric acid. The drug releasing rate is therefore fast. Such structure is helpful for those drugs with low dissolution ability in the gastric acid.

This application claims priority of No. 098114937 filed in Taiwan R.O.C. on May 6, 2009 under 35 USC 119, the entire content of which is hereby incorporated by reference.

### BACKGROUND OF THE INVENTION

1 Field of the Invention

The present invention relates to a space filled drug release structure and a method of manufacturing the same, wherein the drug releasing rate may be controlled, and drug particles are isolatedly and uniformly dispersed in a matrix composed of a highly water-absorbing polymeric material. The release rate of the drug in the structure can be controlled.

2. Related Art

A drug, after being taken into the human body, rapidly enters the stomach. The gastric fluid in the stomach is an extremely acidic liquid with the pH value ranging from 1.0 to 2.5. Most drugs disintegrate in the gastric acid within an extremely short period of time and the effect of drug is consequently released. This rapid dissolution rate can result in fast drug releasing rate. The fast drug releasing rate consumes the drug quickly. The drugs thus have to be taken many times a day so that the disease can be cured. However, it is not so easy for some persons, who are busy or have some special diseases, to take the drugs many times a day. So, it is necessary to lengthen the time for drug release.

Taiwan Patent Publication No. 200833372 discloses a mixture of a drug and insoluble (meth)acrylate copolymers to slow the drug releasing rate in the gastric acid. On the other hand, WO2006094083 discloses a matrix composed of polysaccharide and a gelling agent, and drug particles with 28 to 45 weight percentages are mixed with the matrix. Then, the matrix is gelled to cover the drug particles so that the drug releasing rate may be slowed, and the drug may be released for a long period of time, such as 24 hours. These previous techniques focus on the slowing of the drug releasing rate, and are not related to the increase of the drug releasing rate.

There are many substances having high potential to serve as drugs; however, their release rate are originally very slow. Accordingly, these substances cannot serve as the materials of the drugs. For example, the calcium phosphate material having the scientific name of hydroxyapatite (Ca₁₀(PO₄)₆(OH)₂) is the main inorganic material of the tooth and the bone. The hydroxyapatite has the extremely low solubility in water, and the solubility in acid is also low. According to the report of Chow in the year of 2001 (Chow, L. C., “Calcium phosphate cements.” Octacalcium phosphate, vol. 18, pp. 148-163), the solubility of hydroxyapatite at 37° C. is significantly affected by the pH value of the liquid. Some data of the report are shown and plotted in FIG. 1. As shown in FIG. 1, the solubility of hydroxyapatite is increased as the pH value is decreased. Taking the pH value as 4 and the temperature as 37° C. as an example, the equilibrium solubility of calcium of hydroxyapatite is only equal to 0.025 mol/liter. If the hydroxyapatite is served as the drug, the release of Ca ion into the gastric acid is very time consuming.

Therefore, due to its low solubility, the hydroxyapatite is usually treated as the material which is not suitable to be used as the material of drugs. If this substance has to be served as the material of drugs, it is an important issue to increase its drug releasing rate. If a safe method of increasing the drug releasing rate can be developed, many substances, which cannot originally serve as the materials of drugs, may be used to produce new kinds of drugs.

The hydroxyapatite is the main inorganic substance for the tooth and the bone, and has an extremely low solubility in the sputum and in the blood. Though the solubility of hydroxyapatite is very low, the calcium is released slowly into blood. As the absorption of calcium is slower than the releasing of calcium, even the osteoporosis may be resulted. If the hydroxyapatite can dissolve in the gastric acid to release the calcium, then the hydroxyapatite may be served as the substance for providing the calcium. However, the important issue is to increase the release rate of the hydroxyapatite.

A typical drug is formed by mixing drug particles with a matrix of organic or inorganic excipient, and then pressing or extruding the mixture, wherein the drug particles occupy more than 20 weight percentages. Thus, the drug particles under the weight percentages are connected together. After the drug is taken into the human body, the excipient disintegrates rapidly in the gastric acid, and then the drugs are released quickly. Because the drug releasing starts from the surface, the drug releasing rate of the typical drug particles relates to the effective surface area of the drug particles. The effective surface area represents the contact surface area with the surrounding liquid. Only the surface area in contact with the surrounding liquid can dissolve into the liquid. So, it is an important issue of the present invention to provide a structure, in which the drug particles are separated from one another and contacted effectively with the surrounding liquid.

As the drug releasing rate is slow, extra care has to be taken to monitor the change of drug releasing rate in other liquid. For example, people may drink some beers and wines before or after taking the drug. The release rates of many durgs are much faster in alcohol than those in gastric acid. The sudden increase in drug releasing rate sometimes is very harmful to patients. Therefore, for the structure with long-time releasing capability, the drug releasing rate in alcohol has to be measured as well.

### SUMMARY OF THE INVENTION

It is therefore an objective of the present invention to provide a space filled drug release structure and a method of manufacturing the same, wherein the drug releasing rate may be increased by adding isolatedly and uniformly dispersed drug particles into a matrix composed of a highly water-absorbing polymeric material.

To achieve the above-mentioned objective, the present invention provides a space filled drug release structure including a matrix and a plurality of drug particles. The matrix is composed of a highly water-absorbing polymeric material. The drug particles are dispersed in the matrix. The matrix has a plurality of pores and a plurality of passages interconnecting the pores.

A component of each of the drug particles is selected from a drug group consisting of drug components, which are hardly dissolvable to gastric acid. The component of each of the drug particles may be a ceramic material, such as hydroxyapatite. The highly water-absorbing polymeric material includes at least one material selected from a polymeric group consisting of pectin, carrageen and gelatin etc. For example, the highly water-absorbing polymeric material is agar.

The present invention also provides a method of manufacturing a space filled drug release structure. The method includes a mixing step, a heating step and a shaping step. In the mixing step, a plurality of materials is mixed to form a mixture, wherein the materials include a highly water-absorbing polymeric material, deionized water and a drug material. In the heating step, the mixture is heated. In the shaping step, the mixture is shaped to form the space filled drug release structure containing a matrix and a plurality of drug particles dispersed in the matrix. The matrix has a plurality of pores and a plurality of passages interconnecting the pores.

Therefore, the space filled drug release structure of the present invention and the method of manufacturing the same can effectively solve the problem that the material which cannot originally serve as the material of the drug. Thus, the present invention provides the new use and new application for such material.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention.

FIG. 1 shows the equilibrium solubility of the calcium ions and the phosphoric acid ions from hydroxyapatite at the temperature of 37° C. and different pH values.

FIG. 2 is a pictorial view showing a space filled drug release structure according to the present invention.

FIG. 3 is a schematically partially cross-sectional view showing the space filled drug release structure of FIG. 2.

FIG. 4 shows the morphology of samples used in the first to third examples.

FIG. 5 shows the morphology of samples used in the fourth to sixth examples.

FIG. 6 shows the morphology of samples used in the seventh to twelfth examples.

FIG. 7 is a pictorial view showing another space filled drug release structure according to the present invention.

FIG. 8 is a schematically partially cross-sectional view showing the space filled drug release structure of FIG. 7.

FIG. 9 is an optical micrograph showing space filled drug release structures of the seventh to ninth examples.

FIG. 10 shows the release rate of the drug in the space filled drug release structures in the artificial gastric acid for the first to twelfth examples.

FIG. 11 shows the dissolution properties of the space filled drug release structures in the artificial gastric acid containing 50% alcohol for the thirteenth to eighteenth examples.

FIG. 12 is a flow chart showing a method of manufacturing the space filled drug release structure according to the present invention.

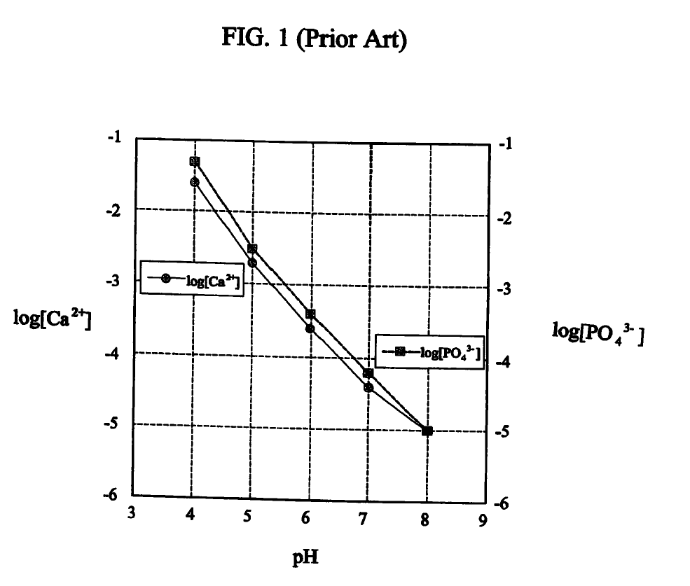

### DETAILED DESCRIPTION OF THE INVENTION

The present invention will be apparent from the following detailed description, which proceeds with reference to the accompanying drawings, wherein the same references relate to the same elements.

The present invention discloses a novel structure with a high effective surface area. In this structure, drug particles are isolatedly and uniformly dispersed within a space, which may also be referred to as a medium or a matrix. To achieve this objective, a volume ratio of the drug particles to this space has to be lower than a threshold value, which may be predicted according to the percolation theory. According to this theory, the volume ratio of the particles to the space has to be lower than 16%. According to the experience of the applicants and the experimental results of the volume ratios of 2% and 3%, the volume ratio of the particles to the space preferably ranges from 0.1% to 10%, and more preferably ranges from 1% to 5% in order to prevent the particles from contacting with one another.

In order to disperse the drug particles within a space, this space may be filled with any other organic or inorganic material so that the drug particles may be dispersed. However, in order to satisfy the edible safety, this spatial matrix material has to satisfy the medicine safety regulations. In addition, in order to make the drug particles be isolatedly and uniformly dispersed within this space for a long time, this spatial matrix material needs to have a lower solubility, and to cover the drug particles, and to allow an surrounding liquid, such as the gastric acid in the stomach, to rapidly enter the spatial matrix material and thus contact with the drug particles.

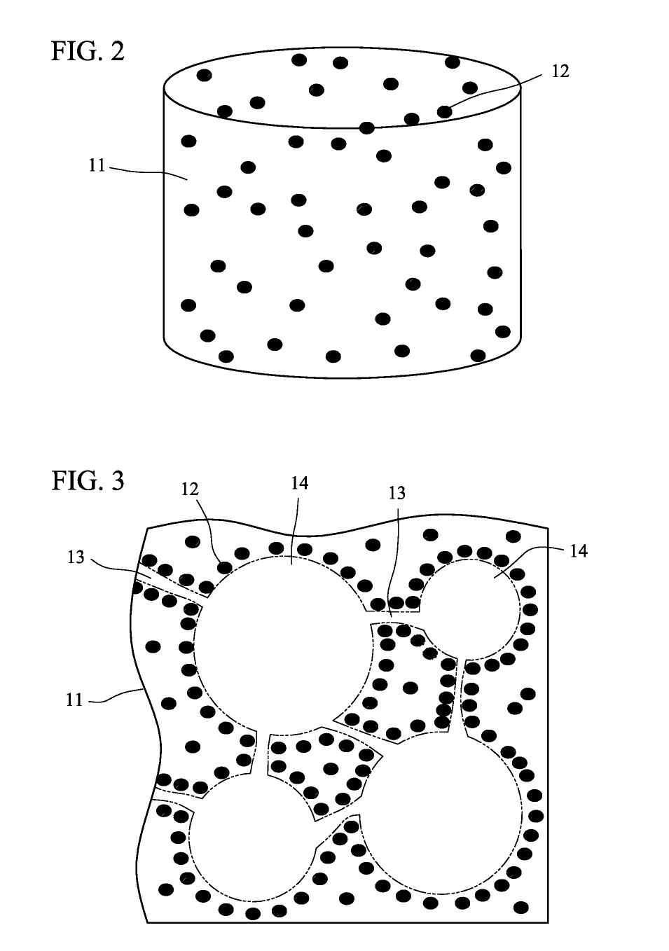

The present invention discloses a space filled drug release structure, which can make the drug particles be isolatedly and uniformly dispersed within a space (also referred to as a medium or a matrix) for a long time, such that a plurality of pores and water passages interconnecting the pores are formed. A little water in these water passages interconnecting one other may exchange with the surrounding liquid, so that the surrounding liquid can enter this space and thus contact with the drug particles. Furthermore, the drug may be released for a long time due to the low solubility of the drug. FIG. 2 is a pictorial view showing a space filled drug release structure according to the present invention. As shown in FIG. 2, the space filled drug release structure includes a matrix 11 and a plurality of drug particles 12. The drug particles 12 are dispersed in the matrix 11, and are preferably isolatedly and uniformly dispersed within this matrix (also referred to as a space or a medium) 11, which is composed of a highly water-absorbing polymeric material. The component of each of the drug particles 12 is selected from a drug group consisting of drug components, which are hardly dissolvable to the gastric acid. For example, the component of each of the drug particles is a ceramic material. In the present invention, the component of each of the drug particles is selected from at least one material of a calcium phosphate group, such as hydroxyapatite.

The highly water-absorbing polymeric material includes at least one material selected from a polymeric group consisting of pectin, carrageen and gelatin etc. In the following examples to be illustrated, the highly water-absorbing polymeric material is agar.

FIG. 3 is a schematically partially cross-sectional view showing the space filled drug release structure of FIG. 2. Referring to FIG. 3, the matrix 11 has a plurality of pores 14 and a plurality of passages 13 interconnecting with the pores 14. The volume ratio of the pores 14 and the passages 13 to the space filled drug release structure is preferably greater than or equal to 16%. The passages 13 may partially or entirely contain water such that a plurality of water passages is formed. Similarly, the pores 14 may also partially or entirely contain water. It is to be noted that the drug particles 12 may exist on the surfaces of the pores 14 and the surfaces of the passages 13, as well as in the matrix 11.

The space filled drug release structure according to the present invention has a long-term drug releasing capability. The novelty and utility of the present invention will be discussed according to the following experimental examples.

Before the experiments are carried out, the artificial gastric fluid has to be prepared. In the following examples, the artificial gastric fluid is prepared according to the specification stated in Chinese Pharmacopoeia, the sixth edition, page 205 (Jeou Chou Book Co., Ltd.), wherein 2.0 grams of salt and 3.2 grams of pepsin (ACROS ORGANICS, USA) are dissolved in 7.0 cc. of hydrochloric acid and a predetermined amount of deionized water so that 1000 cc. of artificially simulated gastric fluid with the pH value of 1.2 is prepared.

### FIRST TO THIRD EXAMPLES

### Control Group

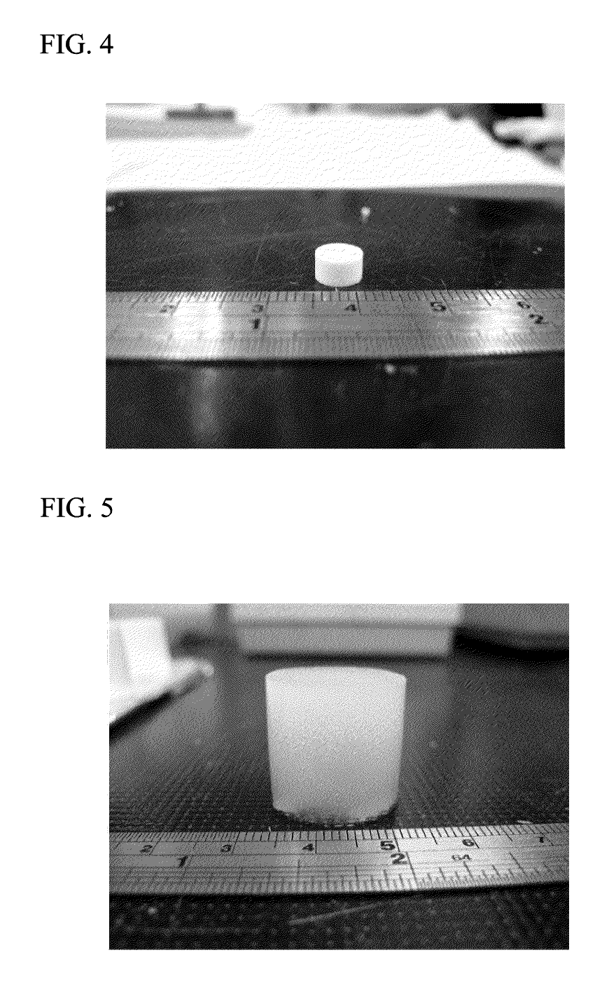

In these examples, hydroxyapatite powder (obtained from Riedel-de Haean Co., Germany) is pressed and shaped to form cylindrical discs with the morphology shown in FIG. 4. The three cylindrical discs (drugs) are respectively disposed into three test tubes, each containing 20 cc. of artificial gastric fluid. These test tubes are placed in a water bath at a temperature of 37° C. Then, the relationship between the pH value and time is recorded, and the results are shown in Table 1. Table 1 shows the initial weight of the hydroxyapatite drug and the variation of the pH value with respect to time for the drug in the artificial gastric fluid. It is to be noted that the cylindrical disc is disintegrated into several pieces after being placed into the artificial gastric fluid for 10 minutes.

TABLE 1

| | First | Second | Third |
|---|---|---|---|
| | example | example | example |
| Weight | 0.129 | 0.111 | 0.128 |
| (grams) | | | |
| Time (hours) | pH value | pH value | pH value |
| 0.2 | 1.26 | 1.24 | 1.20 |
| 0.5 | 1.30 | 1.26 | 1.24 |
| 1 | 1.42 | 1.37 | 1.33 |
| 1.5 | 1.47 | 1.42 | 1.41 |
| 2 | 1.57 | 1.53 | 1.54 |
| 2.5 | 1.66 | 1.61 | 1.62 |
| 3 | 1.74 | 1.69 | 1.72 |
| 4 | 1.82 | 1.78 | 1.77 |
| 5 | 1.88 | 1.82 | 1.81 |
| 6 | 1.93 | 1.87 | 1.86 |
| 7 | 2.03 | 1.95 | 1.96 |
| 8 | 2.14 | 2.05 | 2.03 |
| 21 | 2.19 | 2.11 | 2.14 |

### FOURTH TO SIXTH EXAMPLES

In the following examples, a polymeric matrix material with the highly water-absorbing ability is made from a highly water-absorbing natural organic powder, which is an agar available from Fisher Scientific Co., USA. In addition, the stability of the polymeric matrix material in the artificial gastric fluid is analyzed. First, 200 cc. of deionized water and 10 grams of highly water-absorbing natural polymeric powder (i.e., agar powder) are mixed and stirred uniformly, and then heated in hot water for ten minutes. Then, the heated mixture is poured into a cylindrical polytetrafluoroethylene (PTFE) mold and then cooled. The morphology of the sample is shown in FIG. 5. Then, the cylindrical sample is cut into four pieces, and three of the pieces are respectively placed into three test tubes, each containing the artificial gastric fluid with the weight that is ten times that of the piece. The test tubes are placed in a water bath at a temperature of 37° C., and then the changes of the pH values are recorded, as shown in Table 2. Table 2 shows the initial weight of the highly water-absorbing matrix sample and the variation of the pH value with respect to time for the sample in the artificial gastric fluid.

TABLE 2

| | Fourth | Fifth | Sixth |
|---|---|---|---|
| | example | example | example |
| Sample | 1.694 | 1.350 | 1.577 |
| weight | | | |
| (grams) | | | |
| artificial | 17 | 14 | 16 |
| gastric fluid | | | |
| (/cc.) | | | |
| Time in the | | | |
| artificial | | | |
| gastric fluid | | | |
| (hours) | pH value | pH value | pH value |
| 0.1 | 1.19 | 1.15 | 1.14 |
| 1 | 1.14 | 1.12 | 1.11 |
| 2 | 1.18 | 1.14 | 1.13 |
| 3 | 1.13 | 1.10 | 1.09 |
| 4 | 1.07 | 1.01 | 1.04 |
| 5 | 1.21 | 1.17 | 1.15 |
| 6 | 1.10 | 1.06 | 1.06 |
| 7 | 1.15 | 1.12 | 1.10 |
| 24 | 1.29 | 1.25 | 1.24 |

According to Table 2, it is obtained that 10 grams of agar may be mixed with 200 cc. of water to form a highly water-absorbing polymeric matrix material, as shown in FIG. 5. The highly water-absorbing polymeric matrix may have many bubbles and water passages so that a lot of water may be kept in the matrix. The density of the highly water-absorbing polymeric matrix, which may be determined according to the measured volume and weight, is equal to 1.11±0.01 g/cm³. The highly water-absorbing polymeric matrix is very stable, and does not affect the pH value of the gastric acid after a long period of time (24 hours).

### SEVENTH TO NINTH EXAMPLES

In these three examples, the hydroxyapatite particles are uniformly dispersed in the highly water-absorbing polymeric matrix, and the release rate of each sample in the artificial gastric fluid is measured.

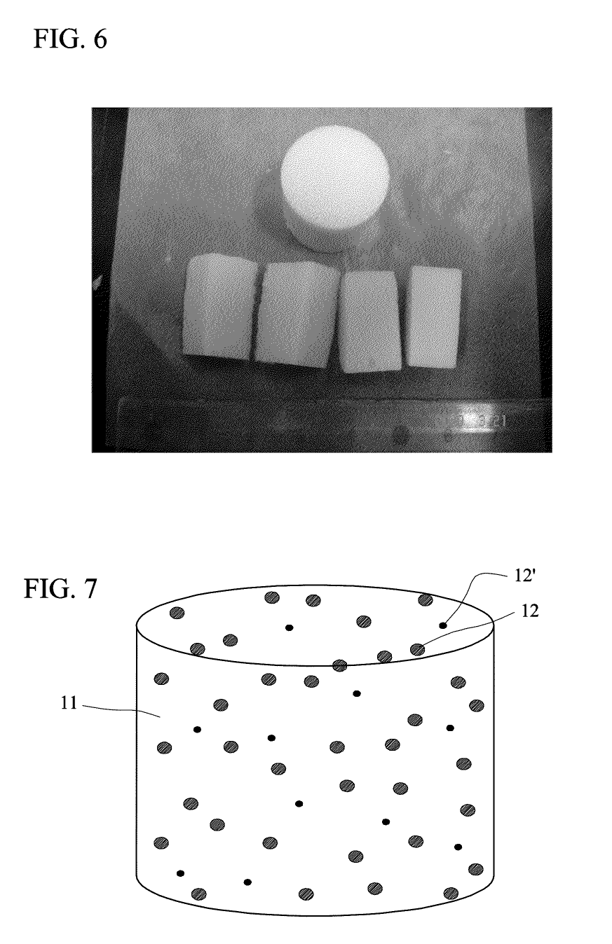

First, 200 cc. of deionized water, 2 cc. of dispersant (ammonium salt of polymethacrylic acid, PMAAN), 10 grams of hydroxyapatite powder and 10 grams of highly water-absorbing natural agar powder are mixed and stirred uniformly, and then heated in hot water bath for ten minutes. Then, the heated mixture is poured into the PTFE mold and then cooled to form cylindrical samples. The cylindrical sample is cut into four pieces, wherein the cylindrical sample and the four pieces are shown in FIG. 6. Three of the pieces are respectively placed into three test tubes, each containing the artificial gastric fluid with the weight that is ten times that of the piece. The weight of the hydroxyapatite powder in each sample is about 0.07 grams.

The test tubes are placed in a water bath with the temperature of 37° C., and then the changes of the pH values are recorded, as shown in Table 3. Table 3 shows the initial weight of the space filled drug release sample and the variation of the pH value with respect to time for the sample in the artificial gastric fluid. In order to verify the stability of the artificial gastric fluid, a reference group, in which the test tube contains only 20 cc. of artificial gastric fluid, is also prepared.

TABLE 3

| | Reference | Seventh | Eight | Ninth |
|---|---|---|---|---|
| | group | example | example | example |
| Initial weight | — | 1.613 | 1.580 | 1.470 |
| (grams) | | | | |
| Weight of | | 0.073 | 0.071 | 0.066 |
| hydroxyapatite | | | | |
| (grams) | | | | |
| Time in the | | | | |
| artificial | | | | |
| gastric fluid | | | | |
| (hours) | pH value | pH value | pH value | pH value |
| 0.5 | 1.16 | 1.36 | 1.35 | 1.36 |
| 2 | 1.14 | 1.63 | 1.57 | 1.58 |
| 4 | 1.09 | 1.88 | 1.79 | 1.78 |
| 6 | 1.12 | 2.07 | 1.97 | 2.01 |
| 9.5 | 1.15 | 2.54 | 2.43 | 2.38 |
| 23 | 1.15 | 3.07 | 2.93 | 2.74 |

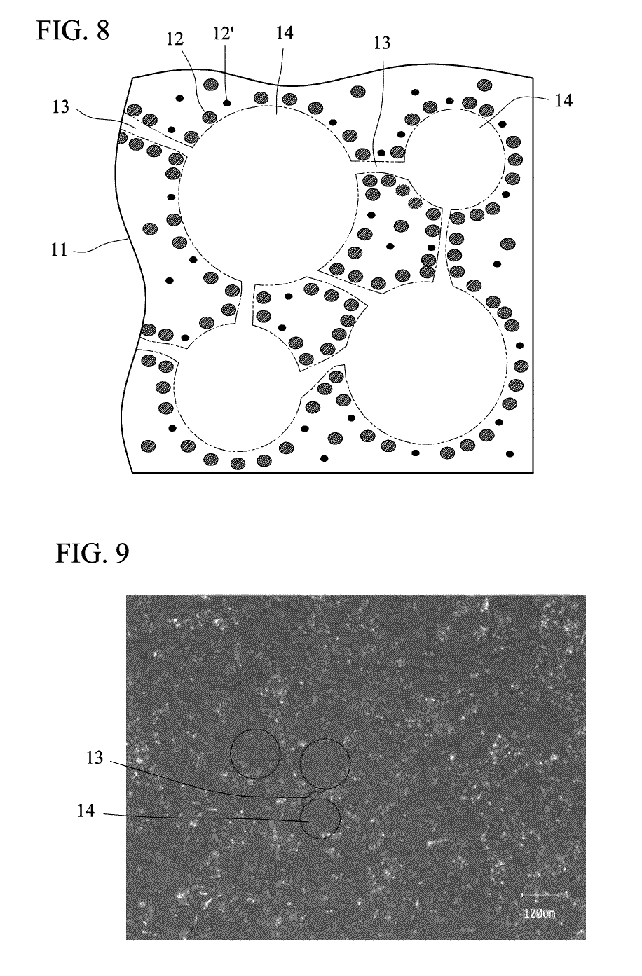

According to the samples containing the hydroxyapatite particles and the highly water-absorbing polymeric matrix in the three examples, the amount of the hydroxyapatite in each of the samples is less than that in each of the hydroxyapatite discs used in the first to third examples. However, the samples of the seventh to ninth examples can slowly increase the pH value of the artificial gastric acid, and thus may serve as the drug for controlling the pH value of gastric acid. The increase of pH value is resulted from the dissolution of hydroxyapatite. This represents that the space filled drug release structure may also serve as a supplement of calcium. This space filled drug release structure may also have two kinds of drug components mixed together. For example, hydroxyapatite and vitamin (e.g., vitamin D3) may be mixed into the space filled drug to enhance the absorption of calcium. As shown in FIGS. 7 and 8, the drug particles contain two different components, so the drug particles 12 and 12′ are dispersed in the matrix 11. The drug particles 12 may be composed of hydroxyapatite, and the drug particles 12′ may be composed of vitamin D3 or other drugs for curing other diseases. It is to be noted that the drug particles 12 and 12′ may be disposed on the surfaces of the pores 14 and the surfaces of the passages 13, as well as in the matrix 11.

According to the space filled drug release structure in the seventh to ninth examples, the dissolution rate of the hydroxyapatite in the artificial gastric fluid is faster than that of the hydroxyapatite discs in the first to third examples. In the seventh to ninth examples, the weight of the hydroxyapatite in the space filled drug release structure is only equal to 0.07 grams, and only occupies 5 weight percentages (5% of weight ratio) of the space filled drug release structure, which is equal to 2 volume percentages (2% of volume ratio). Under the volume percentages, the hydroxyapatite particles are isolatedly dispersed within the matrix of the space filled drug release structure. FIG. 9 is an optical micrograph showing space filled drug release structures of the seventh to ninth examples. As shown in FIG. 9, the pore 14 and the passage 13 are marked, wherein the black portion represents the pore, the white particles are the hydroxyapatite particles, and the gray area represents the agar.

### TENTH TO TWELFTH EXAMPLES

In these three examples, more hydroxyapatite particles are uniformly dispersed within the highly water-absorbing polymeric matrix, and the release rate of each sample in the artificial gastric fluid is measured.

First, 200 cc. of deionized water, 2 cc. of dispersant (PMAAN), 20 grams of hydroxyapatite powder and 10 grams of highly water-absorbing natural agar powder are mixed and stirred uniformly and then heated in the hot water bath for ten minutes. Then, the heated mixture is poured into the cylindrical PTFE mold and then cooled to form cylindrical samples. One cylindrical sample is cut into four pieces. Three of the pieces are respectively placed into the test tubes, each containing the artificial gastric fluid with the weight that is ten times that of the piece. The test tubes are placed in a water bath with the temperature of 37° C., and then the changes of the pH values with time are recorded, as shown in Table 4. Table 4 shows the initial weight of the space filled drug release sample and the variation of the pH value with respect to time for the sample in the artificial gastric fluid. In order to verify the stability of the artificial gastric fluid, a reference group, in which the test tube only contains 20 cc. of artificial gastric fluid, is also prepared.

TABLE 4

| | Reference | Tenth | Eleventh | Twelfth |
|---|---|---|---|---|
| | group | example | example | example |
| Initial | — | 1.980 | 2.272 | 2.364 |
| weight | | | | |
| (grams) | | | | |
| Weight of | — | 0.17 | 0.20 | 0.20 |
| hydroxyapatite | | | | |
| (grams) | | | | |
| Time in the | | | | |
| artificial | | | | |
| gastric fluid | | | | |
| (hours) | pH value | pH value | pH value | pH value |
| 0.5 | 1.16 | 1.34 | 1.36 | 1.43 |
| 2 | 1.14 | 1.54 | 1.57 | 1.65 |
| 4 | 1.09 | 1.83 | 1.82 | 2.02 |
| 6 | 1.12 | 2.00 | 2.01 | 2.25 |
| 9.5 | 1.15 | 2.30 | 2.30 | 2.58 |
| 23 | 1.15 | 2.66 | 2.69 | 3.07 |

The weight percentages of the hydroxyapatite particles contained in the samples of the tenth to twelfth examples are higher than those in the seventh to ninth examples. With the increase of the hydroxyapatite particles, the release rate of hydroxyapatite in the artificial gastric fluid is also increased. In the tenth to twelfth examples, the weight of hydroxyapatite in the space filled drug release structure is only equal to 0.2 grams, and only occupies 8.6 weight percentages, which is equal to 3.0 volume percentages. Under the volume percentages, the hydroxyapatite particles are isolatedly dispersed within the matrix of the space filled drug release structure.

In each of the seventh to twelfth examples, the dispersant is added in order to disperse the drug particles. However, to stir the mixture is also a method for dispersing the drug particles. So, the drug particles may be uniformly dispersed by using dispersant or stirring or both. The dispersant should not influence the dissolution and release of the drug particles. So, 2 cc. of dispersant (ammonium salt of polymethacrylic acid, PMAAN) is added to 15 cc. of artificial gastric fluid, and the variation of the pH value with respect to the time is recorded. The results are listed in Table 5. Table 5 shows the variation of the pH value with respect to time for the dispersant in the artificial gastric fluid. According to Table 5, it is obtained that the pH value varies from 1.10 to 1.25 within 24 hours, which represents that the addition of the dispersant does not affect the pH value. Also, it is proved that the change of pH value of the hydroxyapatite-added space fill structure in the artificial gastric fluid is entirely caused by the dissolution of hydroxyapatite.

TABLE 5

| Time in the artificial | |
|---|---|
| gastric fluid (hours) | pH value |
| 0.5 | 1.16 |
| 2 | 1.13 |
| 4 | 1.15 |
| 6 | 1.10 |
| 9.5 | 1.13 |
| 23 | 1.25 |

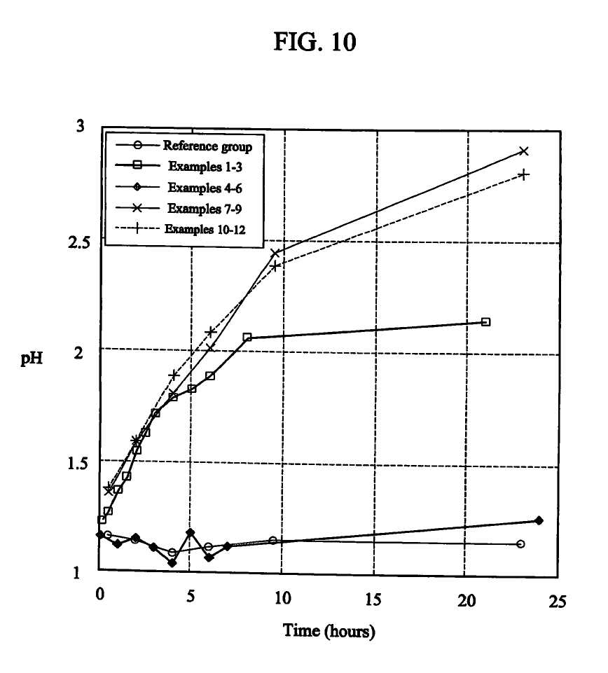

The results of the above-mentioned examples are arranged in FIG. 10, which clearly shows the advantages of the space filled drug release structure of the present invention. In FIG. 10, it shows that the matrix in the space filled drug release structure do not affect the pH value of the artificial gastric fluid, and that the release rate of the hydroxyapatite of the space filled drug release structure in the artificial gastric fluid is faster than that of the disc containing only the hydroxyapatite. Furthermore, the dissolution rate of the hydroxyapatite is a slow one. Taking the values from the Examples 7-9, the pH value changes slowly after a period of 12 hours. The white hydroxyapatite particles are hardly observed within the transparent matrix. Therefore, the dissolution rate of hydroxyapatite (0.07 grams) in the artificial gastric acid (20 cc.) over a period of 12 hours is around 0.004 grams for every cubic centimeter (cc.) of gastric acid. Then, taking the values from the Examples 10-12, most of the white hydroxyapatite particles are dissolved from the transparent matrix over a period of 12 hours. The dissolution rate of hydroxyapatite (0.2 grams) in the artificial gastric acid (20 cc.) over a period of 12 hours is around 0.01 grams for every cubic centimeter (cc.) of gastric acid.

In order to evaluate the drug releasing rate of the present invented structure in alcohol, an artificial gastric acid containing alcohol is prepared. The artificial gastric fluid is prepared by dissolving 2.0 grams of salt and 3.2 grams of pepsin (ACROS ORGANICS, USA) in 7.0 cc. of hydrochloric acid. Then the solution is mixed with a predetermined amount of 50% alcohol and 50% deionized water so that 1000 cc. of an artificially simulated gastric fluid with the pH value of 1.2 is prepared.

### THIRTEENTH TO EIGHTEENTH EXAMPLES

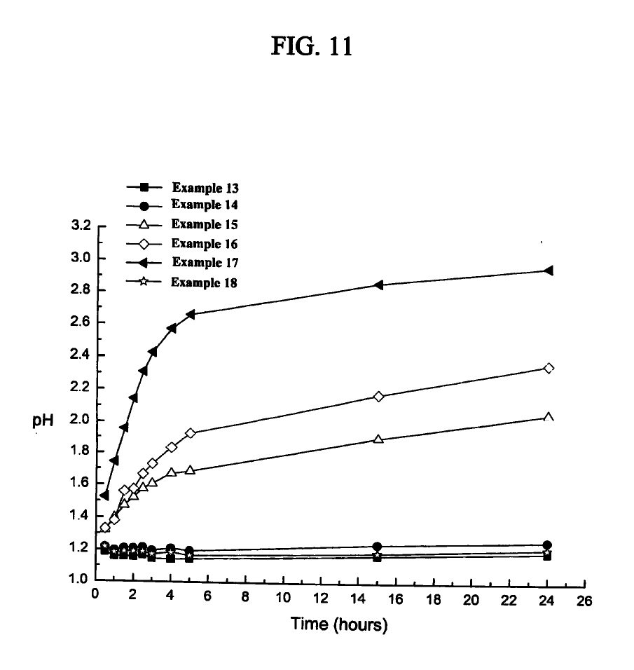

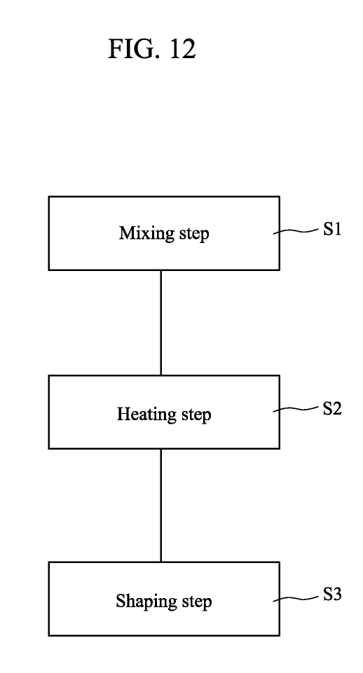

In the thirteenth example, 20 cc. of the artificial gastric acid containing 50% alcohol is added into a test tube. The pH value is monitored against time. The result is shown in Table 6. In the fourteenth example, 2 cc. of dispersant (ammonium salt of polymethacrylic acid, PMAAN) is added into 20 cc. of the artificial gastric acid containing 50% alcohol. The change of pH value with time is also shown in Table 6. In the fifteenth example, the space filled drug release structure composed of the hydroxyapatite particles and agar is prepared with the same procedures as those for the samples of Examples 7-9. The amount of hydroxyapatite in each sample is around 0.07 grams. The matrix is composed of pores and pore channels. In the sixteenth example, the space filled drug release structure composed of the hydroxyapatite particles and agar is mixed with the same procedures as those for the samples of Examples 10-12. The amount of hydroxyapatite in each sample is around 0.2 g rams. In the seventeenth example, only hydroxyapatite powder is used to form a cylindrical disc. The weight of the hydroxyapatite disc is around 0.1 grams. In the eighteenth example, no hydroxyapatite powder is mixed with the agar. The agar sample is prepared with the same procedures as those for the Examples 4-6, and this example can be used as the control sample and treated as the basis for comparison. The samples prepared in the Examples 15-18 are then added separately into the test tubes with 20 cc. of the artificial gastric acid. There is 50% alcohol in the artificial gastric acid. These test tubes were then placed in a water bath at a temperature of 37° C. The change of pH value is shown in Table 6 and FIG. 11.

TABLE 6

| | Thirteenth | Fourteenth | Fifteenth | Sixteenth | Seventeenth | Eighteenth |
|---|---|---|---|---|---|---|
| | example | example | example | example | example | example |
| Time (hours) | pH | pH | pH | pH | pH | pH |
| 0.5 | 1.19 | 1.22 | 1.32 | 1.33 | 1.53 | 1.22 |
| 1 | 1.16 | 1.20 | 1.40 | 1.38 | 1.74 | 1.18 |
| 1.5 | 1.16 | 1.21 | 1.47 | 1.56 | 1.96 | 1.19 |
| 2 | 1.16 | 1.21 | 1.52 | 1.57 | 2.14 | 1.19 |
| 2.5 | 1.17 | 1.22 | 1.58 | 1.67 | 2.31 | 1.19 |
| 3 | 1.15 | 1.20 | 1.61 | 1.73 | 2.43 | 1.17 |
| 4 | 1.14 | 1.21 | 1.67 | 1.83 | 2.57 | 1.19 |
| 5 | 1.15 | 1.20 | 1.69 | 1.93 | 2.66 | 1.17 |
| 15 | 1.17 | 1.24 | 1.90 | 2.18 | 2.86 | 1.19 |
| 24 | 1.19 | 1.26 | 2.06 | 2.36 | 2.96 | 1.22 |

The pH value of the gastric acid containing 50% alcohol (Example 13) is stable within a time period of 24 hours. The agar sample (Example 18) and the dispersant (Example 14) do not affect the pH value of the gastric acid containing alcohol. Comparing FIG. 11 with FIG. 10, the drug releasing rate of the hydroxyapatite in the space fill drug structure (Examples 15 and 16) is not affected much by the presence of the alcohol. It indicates that the release rate of the drug in the structure invented by the present invention is not changed much even when the alcohol is present in the artificial gastric acid. However, the release rate of the hydroxyapatite disc (Example 17) in the artificial gastric acid containing alcohol is much faster than that in the artificial gastric acid without alcohol.

FIG. 11 is a flow chart showing a method of manufacturing the space filled drug release structure according to the present invention. As shown in FIG. 11, the method of manufacturing the space filled drug release structure of the present invention includes the following steps.

Step S1 is a mixing step of mixing a plurality of materials, including the highly water-absorbing polymeric material, the deionized water and the hydroxyapatite material, into a mixture. Similarly, these materials may further include the vitamin, the dispersant or other drugs for curing other diseases. The other drugs for curing other diseases may be released for a long period of time under the protection of the hydroxyapatite material and the highly water-absorbing polymeric material so that the patient may be cured for a long time without taking the drugs several times a day.

Step S2 is a heating step of heating the mixture.

Step S3 is a shaping step of forming the mixture. As mentioned hereinabove, the shaping step is performed by, for example, pouring the mixture into the mold and then cooling the mixture. Thus, the space filled drug release structure contains the matrix and the dispersed drug particles within the matrix.

Consequently, the space filled drug release structure of the present invention and the method of manufacturing the same can effectively solve the problem that the hydroxyapatite material cannot originally serve as the material of the drug. The release rate of the drug in the invented structure is not changed even when the alcohol is added into the artificial gastric acid. The structure is therefore useful to be used a matrix for drug carrier. The present invention also provides the new use and application for the ceramics with low solubility in gastric acid, such as hydroxyapatite.

While the invention has been described by way of examples and in terms of preferred embodiments, it is to be understood that the invention is not limited thereto. To the contrary, it is intended to cover various modifications. Therefore, the scope of the appended claims should be accorded the broadest interpretation so as to encompass all such modifications.

What is claimed is:

1. A space filled drug release structure, comprising:
a matrix composed of a highly water-absorbing polymeric material; and
a plurality of drug particles dispersed in the matrix, wherein the matrix has a plurality of pores and a plurality of passages interconnecting the pores.

2. The space filled drug release structure according to claim 1, wherein a component of each of the drug particles is selected from a drug group consisting of drug components, which the dissolution rate in each cubic centimeter of gastric acid is slower than 0.01 grams over a period of 12 hours.

3. The space filled drug release structure according to claim 2, wherein the component of each of the drug particles is a ceramic material.

4. The space filled drug release structure according to claim 2, wherein the component of each of the drug particles is selected from at least one material of a calcium phosphate group.

5. The space filled drug release structure according to claim 2, wherein the component of the drug particles is hydroxyapatite.

6. The space filled drug release structure according to claim 1, wherein the highly water-absorbing polymeric material comprises at least one material selected from a polymeric group consisting of pectin, carrageen and gelatin.

7. The space filled drug release structure according to claim 6, wherein the highly water-absorbing polymeric material is agar.

8. The space filled drug release structure according to claim 1, wherein a volume ratio of the drug particles to the space filled drug release structure is smaller than 16%.

9. The space filled drug release structure according to claim 1, wherein a volume ratio of the drug particles to the space filled drug release structure ranges from 0.1% to 10%.

10. The space filled drug release structure according to claim 1, wherein a volume ratio of the drug particles to the space filled drug release structure ranges from 1% to 5%.

11. The space filled drug release structure according to claim 1, wherein a portion of the pores contains water.

12. The space filled drug release structure according to claim 1, wherein a volume ratio of both the pores and the passages to the space filled drug release structure is greater than or equal to 16%.

13. The space filled drug release structure according to claim 1, wherein a portion of the passages contains water to form a plurality of water passages.

14. The space filled drug release structure according to claim 1, wherein the drug particles comprise two different components.

15. The space filled drug release structure according to claim 14, wherein one of the components of the drug particles is hydroxyapatite, and the other one of the components of the drug particles is vitamin.

16. A method of manufacturing a space filled drug release structure, the method comprising:
a mixing step of mixing a plurality of materials to form a mixture, wherein the materials comprise a highly water-absorbing polymeric material, deionized water and a drug material;
a heating step of heating the mixture; and
a shaping step for shaping the mixture to form the space filled drug release structure containing a matrix and a plurality of drug particles dispersed in the matrix, wherein the matrix has a plurality of pores and a plurality of passages interconnecting the pores.

17. The method according to claim 16, wherein the materials further comprise vitamin.

18. The method according to claim 16, wherein the materials further comprise a dispersant.

19. The method according to claim 16, wherein the shaping step is performed by pouring the mixture into a mold and cooling the mixture.

20. The method according to claim 16, wherein the highly water-absorbing polymeric material comprises at least one material selected from a polymeric group consisting of pectin, carrageen and gelatin.
